(19) United States
(12) Patent Application Publication
(10) Pub. No.: US 2026/0240551 A1
(43) Pub. Date: Aug. 20, 2026

(54) SURGICAL TEMPLATE AND METHOD FOR CONFIGURING AND/OR MANUFACTURING A SURGICAL TEMPLATE

(71) Applicant: Karl Leibinger Asset Management GmbH & Co. KG, Mühlheim an der Donau (DE)

(72) Inventors: Adem AKSU, Schwenningen (DE); Frank REINAUER, Emmingen-Liptingen (DE); Jens KRIST, Fridingen (DE); Heiner WILD, Dürbheim (DE)

(21) Appl. No.: 19/539,660

(22) Filed: Feb. 13, 2026

(30) Foreign Application Priority Data
Feb. 14, 2025 (DE) 102025105586.6
Feb. 3, 2026 (EP) 26155908.2

(51) Int. Cl. A61B 17/17; A61B 17/16

(52) U.S. Cl. CPC A61B 17/1739; A61B 17/1615; A61B 17/1675; A61B 17/17; A61B 17/1714; A61B 17/1757; A61B 17/1764; A61F 2310/00023

(57) ABSTRACT

A surgical template (OS) for use in the region of a temporal bone, in particular within the framework of performing a radical mastoidectomy, includes a template body (SK) with an outer face (AS) and an inner face (IS) with which the template body (SK) is placeable on the temporal bone. The template body (SK) is penetrated by an opening (D) extending from the outer face (AS) to the inner face (IS). The opening (D) in the template body (SK) is defined with an outer edge (AR), which corresponds to an outer contour of a bone part of the temporal bone (SB) to be removed.

### CROSS-REFERENCE TO RELATED APPLICATION

The present application is related and has right of priority to German Patent Application No. DE 102025105586.6 filed on Feb. 14, 2025, which is incorporated by reference in its entirety for all purposes.

### TECHNICAL FIELD

The invention relates generally to a surgical template for use in the region of a temporal bone, in particular within the framework of performing a radical mastoidectomy. The invention further relates generally to a method for configuring and/or manufacturing a surgical template for use in the region of a temporal bone.

### BACKGROUND

In chronic diseases in the region of the middle ear, surgical treatment in the form of a radical mastoidectomy may be necessary in certain cases. In a radical mastoidectomy on a temporal bone (os temporale), a mastoid (pars mastoidea) is cleaned out, with usually partial removal of the posterior auditory canal wall in order to form an open cavity formed of an attic of the middle ear, the mastoid, and the auditory canal. This cavity is frequently also referred to as the mastoid cavity or the radical cavity. A radical mastoidectomy can be necessary in the case of a pronounced cholesteatoma, which is chronic suppurative otitis media of the middle ear. The aim of the radical mastoidectomy is total removal of a matrix of the cholesteatoma. In operations to be performed in the region of the cranium and, thereby, also the temporal bone, the use of surgical templates to assist the surgeon in performing the operation is also generally known.

CN 215079279 U describes a surgical template in the form of a craniotomy template. This craniotomy template has a template body, which is intended to be placed with an inner face on a temporal bone. On an outer face facing away from the inner face, a contact region for an auricle of a patient is also formed on the template body in order to be able to correctly position the craniotomy template during placement on the temporal bone of the patient. In addition, a viewing window and, adjoining same, a needle guide are also formed in the template body.

### BRIEF SUMMARY

Example aspects of the present invention provide a surgical template, by which a surgeon can be assisted in performing an operation in the region of the temporal bone, preferably in performing a radical mastoidectomy.

According example aspects, a surgical template, which is provided for use in the region of a temporal bone, has a template body with an outer face and an inner face with which the template body is to be placed on the temporal bone. Preferably, the surgical template is provided for use within the framework of a radical mastoidectomy, by which a mastoid cavity, or radical cavity, is surgically formed, for example in order to treat a cholesteatoma.

The surgical template according to example aspects of the invention has a template body, which is configured for placement on a temporal bone (os temporale). When the surgical template is used, this template body is to be placed with the inner face of the template body on the temporal bone, wherein this is to be carried out prior to the operation to be performed on the temporal bone, i.e. in particular prior to performing the radical mastoidectomy.

Within the context of example aspects of the invention, the template body has preferably been formed within the framework of an additive manufacturing process, wherein this has preferably taken place by a 3D printing process.

Preferably, the template body is formed of a plastic, which is preferably a polymer and, more preferably, a thermoplastic, such as polyamide (PA) or polyether ether ketone (PEEK). Alternatively, the template body can also be formed of a metal material, such as, in particular, titanium or stainless steel, or a metal alloy, preferably a titanium alloy. The template body could also be formed of different materials in order to obtain locally different properties, for example locally different rigidities. The template body can be formed in one piece or in multiple pieces.

According to example aspects, the template body is penetrated by an opening extending from the outer face to the inner face. The opening in the template body is defined with an outer edge, which corresponds to an outer contour of a bone part of the temporal bone to be removed. In other words, the template body is therefore equipped with an opening, which extends from the outer face of the template body to the inner face of the template body and passes completely through the template body. This opening is formed with an outer edge in the template body, wherein the outer edge is identical to an outer contour of a bone part to be removed from the temporal bone.

A surgical template configured in such a way has the advantage that it is made precisely clear to a surgeon, via the opening, which bone part in the region of the temporal bone is to be removed. Via the outer edge of the opening, in addition, a guide is formed, along which the surgeon performing the operation can move with a surgical tool, in particular a bone saw. As a result, precise removal of the bone part is possible. Overall, an operation to be performed in the region of the temporal bone can thus be made safer. In particular, a radical mastoidectomy can thus be reliably performed.

The opening in the surgical template preferably has a closed outer edge, in that the opening is completely surrounded by material of the template body in an island-like manner. Preferably, the opening forms a sawing template, in that the outer contour of a bone part to be removed by sawing is defined by the outer edge of the opening. During use, the opening defines in particular an outer contour of a bone part of a mastoid (pars mastoidea) and/or of a tympanic part (pars tympanica) of the temporal bone to be removed.

According to one possible example embodiment of the invention, the outer edge is at least partially formed by an inner contour of a guide portion. By providing the guide portion, the inner contour of which defines the outer edge of the opening, guiding a surgical tool along during removal of the bone part is simplified. Preferably, the guide portion is designed to project toward the outer face with respect to the remainder of the template body in order to ensure better guidance.

According to an additional possible example embodiment of the invention, the surgical template is provided with an attachment portion on which through-openings extending from the outer face to the inner face are formed. These through-openings are used to guide one attachment mechanism through in each case to attach the surgical template to the temporal bone, wherein this is preferably an attachment to a mastoid of the temporal bone. This has the advantage that, due to the attachment, the surgical template can be reliably prevented from slipping while the operation is performed. In particular, bone screws can be guided through the formed through-openings.

When the two above-described example variants are combined, the attachment portion at least partially peripherally surrounds the guide portion, wherein the guide portion on the template body projects toward the outer face with respect to the attachment portion. As a result, a surrounding attachment of the surgical template to the bone part to be removed can be achieved. In particular, the attachment portion extends a smaller distance to the outer face in comparison with the guide portion.

Alternatively, the guide portion can steplessly transition into the attachment portion, such that the guide portion does not form an elevated guidance for the saw blade. As a result, the surgical template can be designed to be more compact. An elevated guidance for the saw blade is not required in numerous cases due to the low wall thickness of the temporal bone.

According to an example embodiment of the invention, the template body has a cover portion, which forms a partition. The partition separates the outer face and the inner face from one another in the region of the cover portion and is provided for at least predominant coverage of an auditory canal by the surgical template. By the surgical template, inadvertent injury to the auditory canal during the operation can thus be effectively prevented. This is the case because, when the surgical template is in place, the auditory canal, in particular the auditory canal of the middle ear, is covered via the cover portion of the surgical template, whereby slipping into the auditory canal or any other inadvertent injury to the auditory canal caused by a surgical tool is ruled out. More preferably, the cover portion is designed to cover the entire auditory canal. In particular, the cover portion is configured in the shape of a cap, i.e. the cover portion preferably has a shape oriented to a protective cap. Preferably, the partition also forms, with an edge, a portion of the outer edge of the opening, such that the cover portion of the template body then adjoins the opening. As a result, the cover portion effectively prevents, via the partition, inadvertent injury to the auditory canal while work is being performed in the opening using a surgical tool to remove the bone part.

In a development of the aforementioned example embodiment, the cover portion is curved toward the inner face and used for insertion into the auditory canal. The cover portion forms not only the partition but also a side wall, which projects toward the outer face with respect to the partition. Advantageously, the surgical template can therefore be inserted together with the cover portion into the auditory canal and thus particularly reliable protection of the auditory canal can be achieved. In addition, the side wall also provides lateral protection. In particular, the curved cover portion is curved in a scoop-like manner. The inner wall defined in the cover portion can be provided and/or configured to be placed at least partially against an inner wall of the auditory canal. Preferably, the side wall at least partially peripherally surrounds the partition of the cover portion.

Further alternatively or additionally, the cover portion of the template body is provided at an end with which the template body is tilted toward the inner face. In this case, the template body is therefore tilted toward the inner face in sections, namely with the end. The cover portion defines a portion of the outer edge of the opening by an edge of the partition. Preferably, the cover portion forms a surface on the partition facing the outer face, which is used for placement of a surgical tool to be used to remove the bone part of the temporal bone and defines a resection depth. This has the advantage that the cover portion of the surgical template therefore also forms a reference for the depth of the bone to be removed. Thus, the surgeon performing the operation can place the instrument on the cover portion and, starting therefrom, perform the resection of the bone part. The end has been tilted in particular with respect to an attachment portion provided to attach the surgical template to the temporal bone.

When the preceding example variant is combined with the possible example embodiment of the invention, according to which the outer edge of the opening is at least partially formed by an inner contour of the guide portion, this formation of the outer edge of the opening is particularly preferably implemented remote from the tilted end. As a result, the surgeon can work laterally into bone to be removed, within the framework of the operation, starting from the surface of the cover portion.

According to an additional possible example embodiment of the invention, at least one drill guide portion is also formed on the template body, which forms at least one drill hole extending from the outer face to the inner face and is used to guide a bone drill for removing a bone part of the temporal bone in each case. As a result, the surgical template can also assist a surgeon in removing a bone part of the temporal bone by drilling, whereby parts of the temporal bone extending in particular in the depth direction can be reliably removed. The bone part to be removed using the bone drill is preferably a part of a tympanic part (pars tympanica) of the temporal bone.

In a development of the aforementioned example embodiment, the at least one drill guide portion can be provided with one guide region each at least on an end situated on the outer face, in which guide region an inner diameter of the drill hole is approximated to an outer diameter of the bone drill. As a result, suitable guidance of the bone drill on the surgical template for the operating surgeon is achieved. More preferably, each guide region is formed by a guide sleeve, which is introduced into the template body on the at least one guide portion. The latter is achieved in particular when the template body is made of a polymer, wherein the introduced guide sleeve is then formed from a metal material. Advantageously, greater strength can thus be achieved in the region of the respective drill guide portion.

Alternatively or in addition to the aforementioned example development, a stop surface is formed on the at least one drill guide portion flush against an opening in the drill hole on the outer face, which stop surface forms a stop for a surgical tool guiding the bone drill. A length of the respective drill hole corresponds to a drill depth which is to be achieved via the bone drill on the inner face when the surgical tool has reached the respective stop surface. This has the advantage that an unambiguous drill depth can be defined by the formed stop and as a result, drilling that is too deep can be reliably ruled out.

More preferably, a plurality of drill guide portions is formed on the surgical template. As a result, the surgical template thus defines a plurality of bone parts to be removed using a bone drill.

When the example embodiment of the guide portion is combined with the example embodiment of the at least one drill guide portion, the at least one drill guide portion is formed on the guide portion adjoining the inner contour. As a result, a bone part to be removed is defined by the opening enclosed by the guide portion and a further bone part, adjoining same, to be removed is defined by the at least one drill guide portion.

According to a possible example embodiment of the invention, the template body is designed in a patient-specific manner. Advantageously, the surgical template is therefore specifically tailored to the patient. In particular, at least the course of the outer edge of the opening, the size of the opening, the position of the opening on the template body and, optionally, the position of the at least one drill guide portion on the surgical template are adapted to the patient. Alternatively, the template body can be shaped in a non-patient-specific manner. In this case, the template body has a pre-defined standard shape, which corresponds to a typical human anatomy. The template body can be provided in different sizes for this purpose.

In a development of the aforementioned possible example embodiment, the template body can be designed having a contact contour on the inner face, which contact contour is configured at least approximately as a counterpart to a surface contour of the temporal bone in the placement region. As a result, the template body can be placed on the temporal bone in a suitable manner.

In particular, the region in which the surgical template is to be placed onto the temporal bone has been captured in advance for the patient-specific design of the surgical template within the framework of a medical imaging process, for example by CT.

Example aspects of the invention also relate to a method for configuring and/or manufacturing a surgical template for use in the region of a temporal bone. A template body is configured with an outer face and an inner face with which the template body is to be placed on the temporal bone. An opening is provided in the template body, which extends from the outer face to the inner face, wherein an outer edge is defined on the opening, which corresponds to an outer contour of a bone part of the temporal bone to be removed.

Within the context of the method according to example aspects of the invention, a configuration or a manufacture, or both a configuration and a manufacture, of a surgical template is/are therefore carried out. A “configuration” is to be understood to mean in particular planning and designing the surgical template, i.e. creating a design or a template for manufacturing the surgical template. “Manufacture” is intended to mean production of the surgical template, i.e. the physical embodiment. In particular, both the configuration and the manufacture of a surgical template are carried out within the framework of the method according to example aspects of the invention. Manufacture is preferably carried out within the framework of an additive manufacturing process, more preferably within the framework of a 3D printing process.

Advantageously, a surgical template can thus be configured and/or manufactured, the use of which makes it precisely clear to a surgeon, which bone part in the region of the temporal bone is to be removed. In addition, a guide can be formed via the outer edge of the opening, along which the surgeon performing the operation can move with a surgical tool, in particular a bone saw. As a result, precise removal of the bone part is possible. Overall, an operation to be performed in the region of the temporal bone can thus be made safe. Preferably, a surgeon can thus be assisted in performing a radical mastoidectomy.

Preferably, the surgical template is also formed with respect to configuration and/or with respect to creation according to at least one of the above-described example variants of a surgical template.

According to a further possible example embodiment of the method according to the invention, a temporal bone implant is also planned and/or manufactured together with the surgical template. Advantageously, this temporal bone implant can then be used after the radial mastoidectomy is performed in order to cover a void in the temporal bone formed by removing the bone part.

### BRIEF DESCRIPTION OF THE DRAWINGS

An advantageous example embodiment of the invention, which is explained in the following, is represented in the drawings, wherein:

FIG. 1 shows a perspective representation of a surgical template according to one example embodiment;

FIG. 2 shows a perspective view of a part of a cranium in the region of a temporal bone with a marked region of the temporal bone to be removed;

FIG. 3 to 6 show further perspective representations of the region of the cranium after placement of the surgical template from FIG. 1;

FIG. 7 shows a further perspective representation of the region of the cranium after removal of the region of the temporal bone;

FIG. 8 shows a further view of the cranium after insertion of a temporal bone implant;

FIGS. 9 to 11 show different views of a surgical template according to a further possible embodiment of the invention; and

FIGS. 12 to 13 show representations of a region of a cranium after placement of the surgical template from FIGS. 9 to 11.

### DETAILED DESCRIPTION

Reference will now be made to embodiments of the invention, one or more examples of which are shown in the drawings. Each embodiment is provided by way of explanation of the invention, and not as a limitation of the invention. For example, features illustrated or described as part of one embodiment can be combined with another embodiment to yield still another embodiment. It is intended that the present invention include these and other modifications and variations to the embodiments described herein.

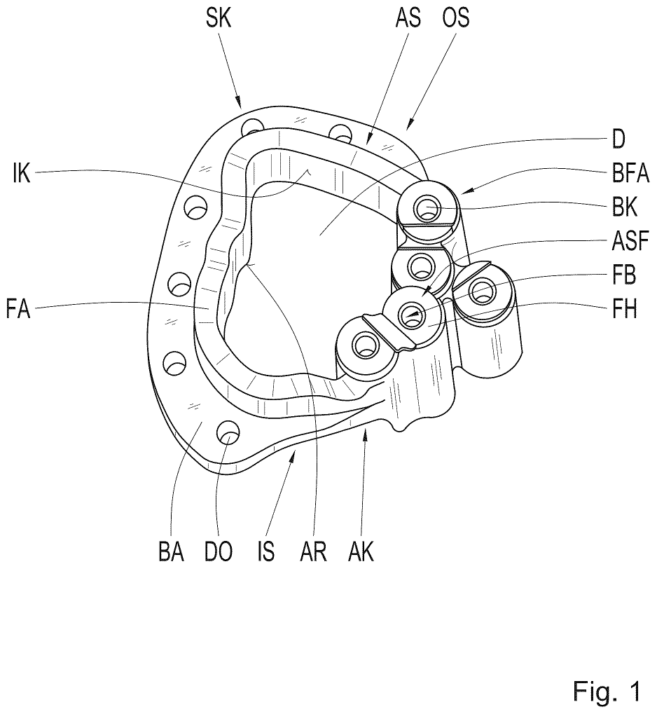

FIG. 1 shows a perspective representation of a surgical template OS, which is designed according to an example embodiment of the invention. The surgical template OS has a template body SK, which is configured on an inner face IS for placement on a temporal bone. The template body SK is penetrated by an opening D, in that the opening D in the template body SK extends from the inner face IS to an outer face AS arranged opposite the inner face IS.

The opening D has an outer edge AR, which is defined on the surgical template OS by an inner contour IK of a guide portion FA of the template body SK. The opening D is designed in the template body SK in the form of an island, in that the guide portion FA completely encloses the opening D and the outer edge AR is defined to be closed by the inner contour IK.

On the outside, the guide portion FA is partially peripherally surrounded by an attachment portion BA, which is provided with a plurality of through-openings DO, wherein the through-openings DO each extend from the inner face IS to the outer face AS. The guide portion FA projects with respect to the attachment portion BA toward the outer face AS of the template body SK.

In addition, a plurality of drill guide portions BFA adjoining the passage D is formed on the guide portion FA, each of which forms a drill hole BK extending continuously from the outer face AS to the inner face IS. The drill guide portions BFA are designed, in part, with different extents between the inner face IS and the outer face AS, whereby different lengths of the drill holes BK are obtained.

The template body SK of the surgical template OS has been manufactured from a polymer within the framework of an additive manufacturing process, wherein the manufacture has preferably taken place within the framework of a 3D printing process. The polymer that has been used is in particular a thermoplastic, such as polyethylene (PE) or polyether ether ketone (PEEK). Guide sleeves FH have been introduced into the template body SK at the openings in the drill guide portions BFA on the outer face, wherein these guide sleeves FH consist in particular of a metal material, for example titanium.

The guide sleeves FH form a guide region FB on each associated drill guide portion BFA, in which an inner diameter of the drill hole BK is reduced over the length of the guide sleeve FH. In addition, a stop surface ASF is defined via the guide sleeves FH on the outer face AS in each case, which stop surface lies flush against the mouth of the drill hole BK.

In addition, the template body SK is provided with a contact contour AK—which is only partially apparent in FIG. 1—on the inner face IS.

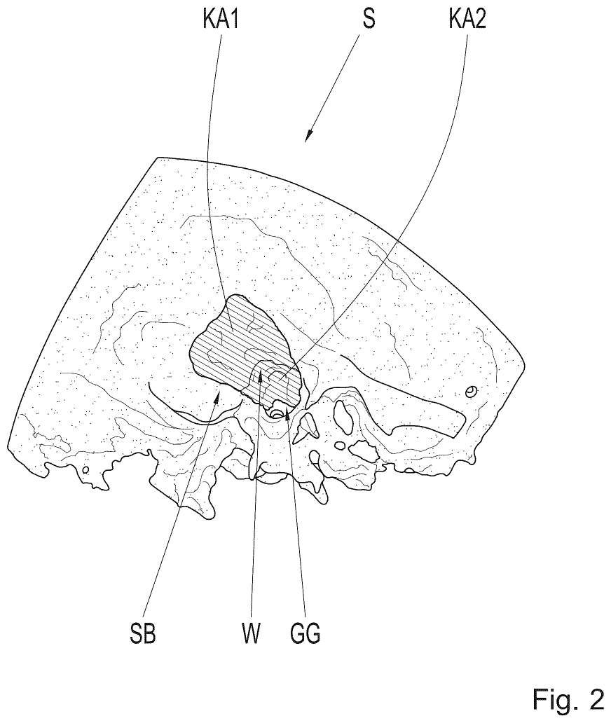

The surgical template OS represented in FIG. 1 has been configured and manufactured for the actual, patient-specific application in the region of a temporal bone SB of a cranium S. To this end, the shape of this cranium S was determined in advance by a medical imaging process, in particular by CT, taking into account a 3D model of the cranium S partially represented in each of FIGS. 2 to 8. The surgical template OS is formed in a patient-specific manner by configuring the opening D and the drill guide portions BFA on the surgical template OS in accordance with bone portions KA1 and KA2, which have been declared regions of the temporal bone SB of the cranium S to be removed within the framework of a radical mastoidectomy and are indicated with shading in FIG. 2. The radical mastoidectomy is to be performed in particular in order to be able to totally remove a cholesteatoma in the region of an external auditory canal GG. The contact contour AK of the template body SK is configured at least approximately as a counterpart to a surface contour of the temporal bone SB in the region surrounding the bone portions KA1 and KA2.

In the present example case, the bone portion KA1 to be removed is a portion of a mastoid (pars mastoidea) of the temporal bone SB, whereas the bone portion KA2 to be removed is a portion of a tympanic part (pars tympanica) of the temporal bone SB and forms a part of the posterior osseus wall W of the external auditory canal GG.

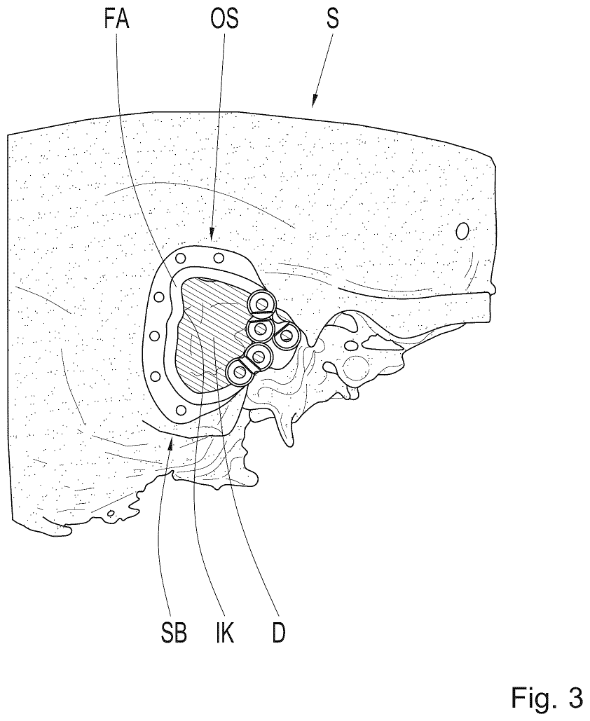

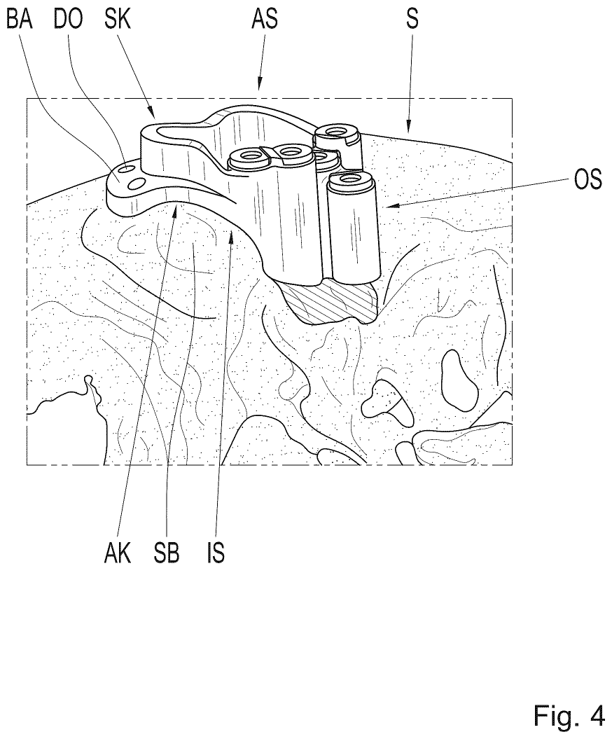

FIG. 3 through FIG. 6 show the surgical template OS in the state placed onto the temporal bone SB, wherein the placement of the template body SK on the temporal bone SB with the contact contour AK formed on the inner face IS is apparent in particular in FIG. 4. The surgical template OS is attached to the temporal bone SB via the attachment portion BA, wherein, for this purpose, in particular bone screws are guided through the through-openings DO for screwing the surgical template OS to the temporal bone SB.

Via the passage D in the surgical template OS, an operating surgeon is shown the region of the bone portions KA1 and KA2 to be removed by a bone saw, wherein the surgeon can move along the inner contour IK of the guide portion FA while working with the bone saw.

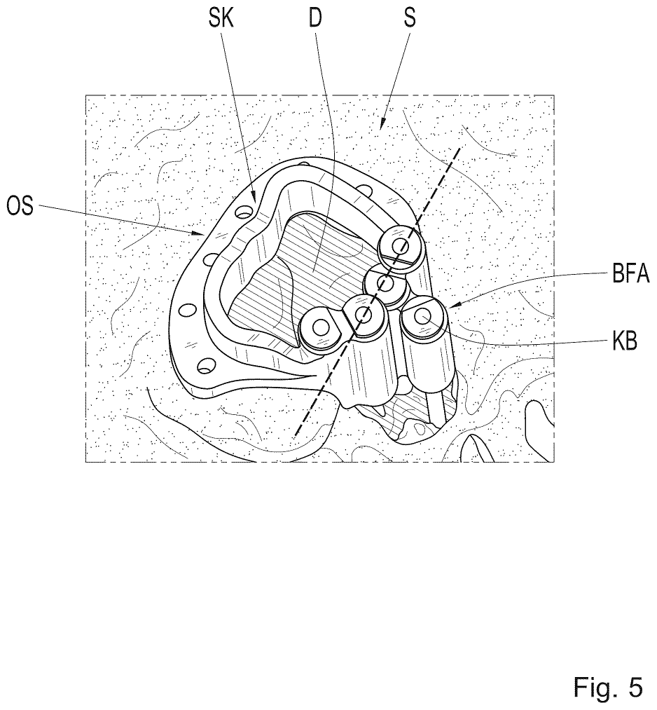

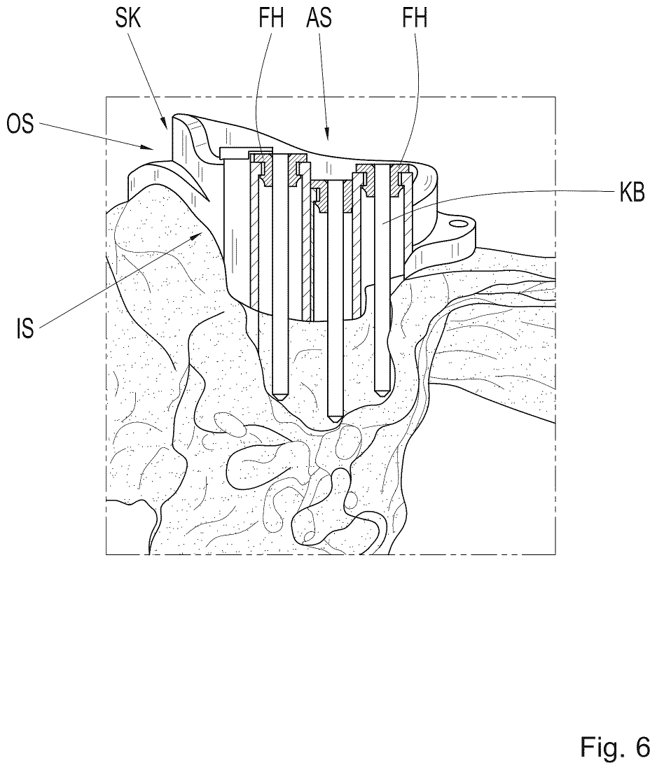

Further regions of the bone portions KA1 and KA2 to be removed are also defined on the surgical template OS via the drill holes BK in the drill guide portions BFA, wherein each drill hole BK is used to guide a bone drill KB through in order to remove a bone part. The bone drill KB is indicated in the representations in FIG. 5 and FIG. 6. In FIG. 6, the guide sleeves FH introduced into the template body SK on the outer face AS are also apparent, wherein the inner diameters, which are defined via the guide regions FB of the guide sleeves FH, are approximated to an outer diameter of the bone drill KB. As a result, guidance of the bone drill KB in the drill guide portion BFA is realized.

By the stop surfaces ASF and the different lengths of the drill holes BK, different drill depths of the bone drill KB are defined on the inner face IS. This is apparent in FIG. 6. In order to achieve the respective drill depth, the operating surgeon must guide the surgical tool guiding the bone drill KB up against the respective stop surface ASF.

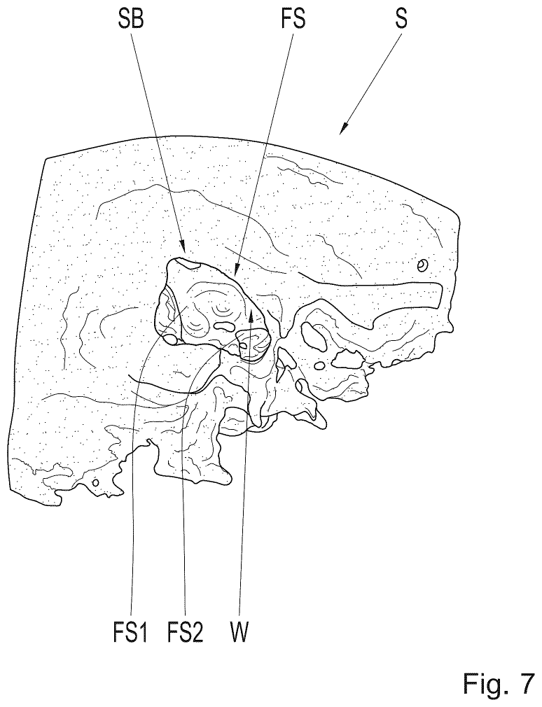

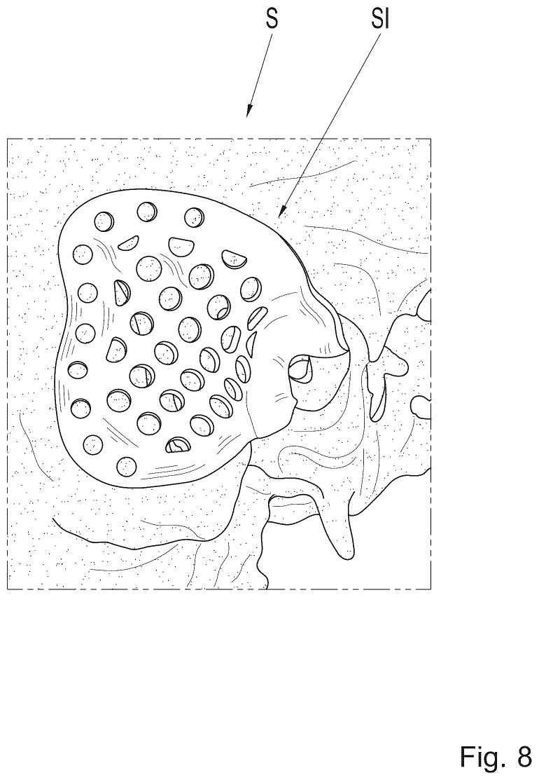

Furthermore, the cranium is shown in FIG. 7 after removal of the bone portions KA1 and KA2, whereby a void FS was formed in the temporal bone SB. Due to the fact that the bone portion KA1 forms a portion of the mastoid and the bone portion KA2 forms a portion of the tympanic part, the void FS has a void part FS1 in the region of the mastoid and a void part FS2 in the region of the tympanic part, wherein, due to the latter, a portion of the wall W has been removed. To cover this void FS, a temporal bone implant SI is attached to the temporal bone SB, as is represented in FIG. 8. This temporal bone implant SI has been configured in a patient-specific manner together with the surgical template OS on the basis of the determined shape of the cranium S.

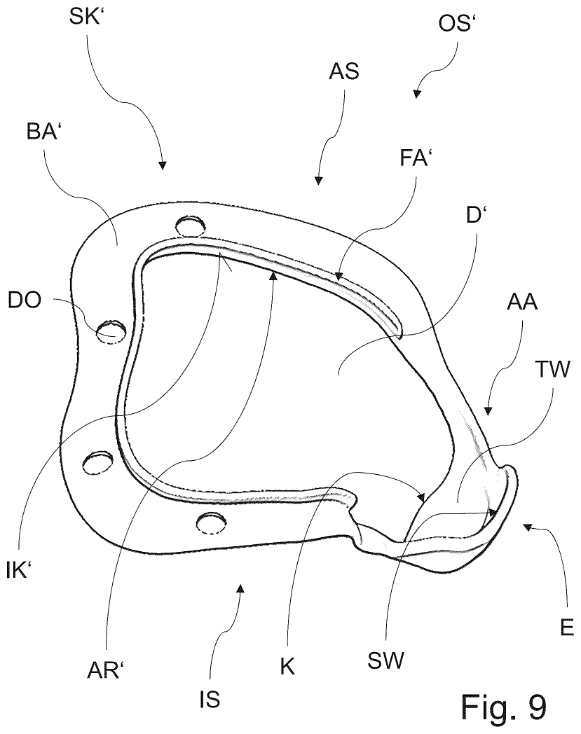

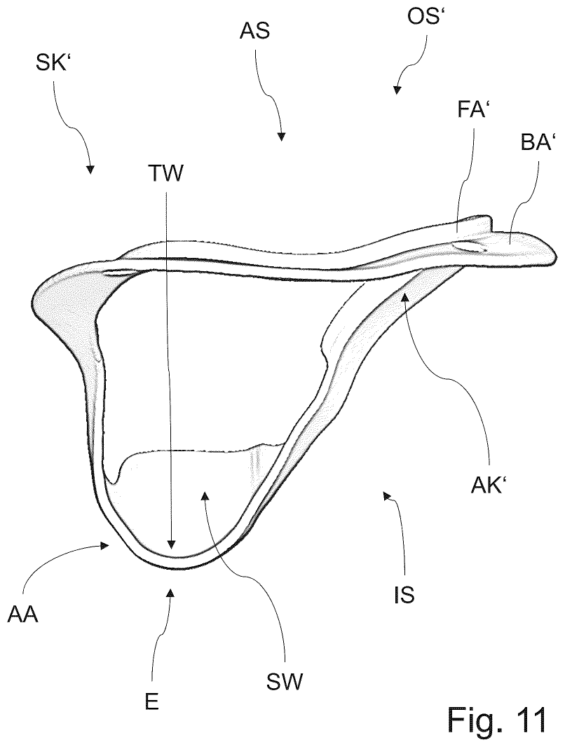

Furthermore, FIGS. 9 to 11 show different views of a surgical template OS′ according to one other possible example embodiment of the invention. This surgical template OS′ is similar to the example variant according to FIG. 1 and is also provided for use in the region of a temporal bone and in particular for performing a radical mastoidectomy.

The surgical template OS′ is once again configured in a patient-specific manner and has a template body SK′, which, in contrast to the example variant according to FIG. 1, is formed of metal and in particular of titanium or a titanium alloy. Alternatively, the surgical template OS′ can be made of plastic. The template body SK′ is configured on an inner face IS to be placed onto the temporal bone and—as is apparent in particular in FIG. 9—is provided with an opening D′, which penetrates the template body SK′ from the inner face IS to an oppositely situated outer face AS. Furthermore, the template body SK′ is provided with a contact contour AK′ on the inner face IS in sections, which contact contour is configured within the framework of the patient-specific configuration of the surgical template OS′ at least approximately as a counterpart to a surface contour of the associated temporal bone in the placement region.

In the template body SK′, the opening D′ is designed in the form of an island and, to this end, is enclosed all around by an outer edge AR′, which is defined in sections by an inner contour IK′ of a guide portion FA′ and in sections by an edge K of a cover portion AA. In the region of the guide portion FA′, the template body SK′ is also provided with an attachment portion BA′, which peripherally surrounds the guide portion FA′ and has through-openings DO. Similarly to the example variant according to FIG. 1, these through-openings DO are provided for guiding bone screws through, via which the surgical template OS′ can be attached to the temporal bone in an intended position. The through-openings DO extend between the inner face IS and the outer face AS. In addition, the guide portion FA′ projects toward the outer face AS with respect to the attachment portion BA′.

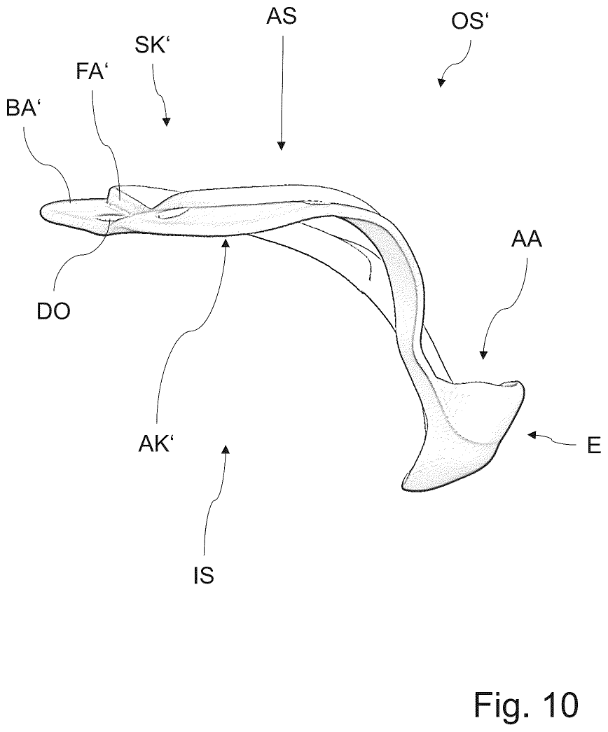

As is apparent in particular with reference to FIG. 10, the cover portion AA is formed at one end E of the template body SK′, with which end E the template body SK′ is tilted toward the inner face IS with respect to the attachment portion BA′. The cover portion AA is curved in a scoop-like manner toward the inner face IS and, with this curvature, forms a partition TW and a side wall SW. Via the partition TW, a local division between the inner face IS and the outer face AS adjoining the opening D′ is implemented. The side wall SW surrounds the partition TW and projects toward the outer face AS of the template body SK′ with respect to the partition TW.

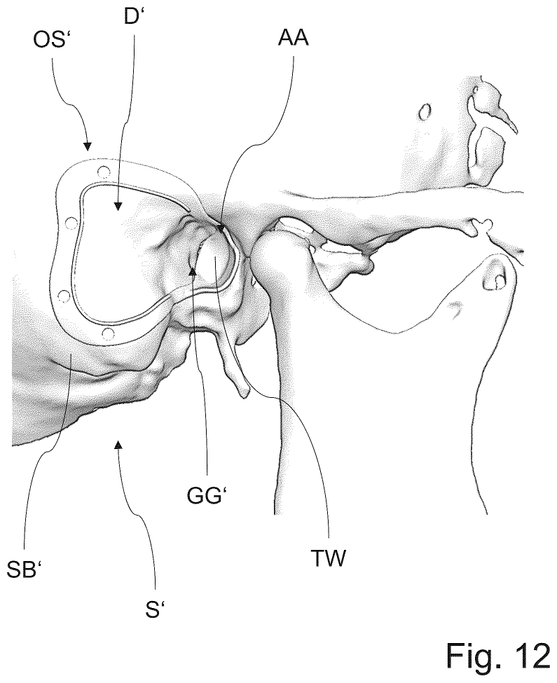

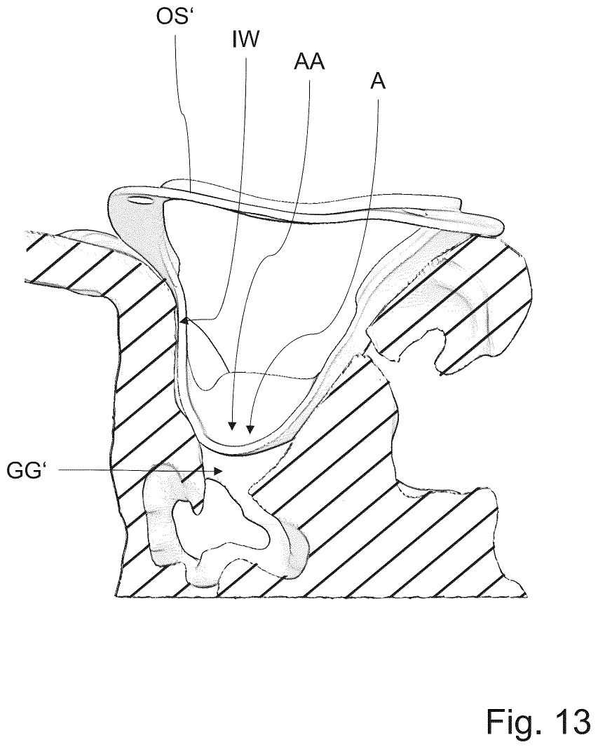

FIG. 12 and FIG. 13 show the surgical template OS′ according to FIGS. 9 to 11 in a state in which the surgical template OS′ specifically configured for this purpose is placed onto a temporal bone SB′ of a cranium S′. As is apparent in FIG. 12 in particular, an auditory canal GG′ is totally covered by the partition TW of the cover portion AA, wherein the surgical template OS′ is also inserted with the cover portion AA into the auditory canal GG′. This is apparent in particular with reference to FIG. 13, wherein it is also apparent that the cover portion AA partially lies against an inner wall IW of the auditory canal GG′ in the attached state of the surgical template OS′.

The bone part to be removed is identified on the temporal bone SB′ by the opening D′ in the attached surgical template OS′. Injury to the auditory canal GG′ is also effectively prevented via the cover portion AA, in that the partition TW prevents slipping into the auditory canal GG′ and the side wall SW protects the inner wall IW of the auditory canal GG′ to be retained.

Furthermore, a surface A is formed by the partition TW, on which the surgeon performing the operation can place the surgical tool and by which a resection depth is defined. Thus, the operating surgeon can work laterally into the bone to be removed, for example, using a bone cutter, starting from the surface A. To this end, the template body SK′ of the surgical template OS′ is then not provided with the guide portion FA′ also in the region of the tilted end E, along which guide portion the operating surgeon can otherwise move with the surgical tool.

By the embodiments according to the invention, a surgical template can be provided, by which a surgeon can be assisted when performing an operation in the region of the temporal bone, preferably a radical mastoidectomy.

Modifications and variations can be made to the embodiments illustrated or described herein without departing from the scope and spirit of the invention as set forth in the appended claims. In the claims, reference characters corresponding to elements recited in the detailed description and the drawings may be recited. Such reference characters are enclosed within parentheses and are provided as an aid for reference to example embodiments described in the detailed description and the drawings. Such reference characters are provided for convenience only and have no effect on the scope of the claims. In particular, such reference characters are not intended to limit the claims to the particular example embodiments described in the detailed description and the drawings.

### LIST OF REFERENCE CHARACTERS

OS, OS′ surgical template
SK, SK′ template body
IS inner face
D, D′ opening
AS outer face
AR, AR′ outer edge
IK, IK′ inner contour
FA, FA′ guide portion
BA, BA′ attachment portion
DO through-openings
BFA drill guide portions
BK drill hole
FH guide sleeves
FB guide region
ASF stop surface
AK, AK′ contact contour
SB, SB′ temporal bone
S, S′ cranium
KA1, KA2 bone portions
GG, GG′ auditory canal
W wall
KB bone drill
FS void
FS1, FS2 void parts
SI temporal bone implant
K edge
AA cover portion
E end
TW partition
SW side wall
IW inner wall
A surface

1-19. (canceled)

20. A surgical template (OS; OS′) for use proximate a temporal bone (SB; SB′), the surgical template (OS; OS′) comprising:
a template body (SK; SK′) with an outer face (AS) and an inner face (IS), the template body (SK; SK′) placeable on the temporal bone at the inner face (IS),
wherein the template body (SK; SK′) is penetrated by an opening (D; D′) extending from the outer face (AS) to the inner face (IS), and the opening (D; D′) is defined with an outer edge (AR; AR′) that corresponds to an outer contour of a bone part of the temporal bone (SB; SB′) to be removed.

21. The surgical template (OS; OS′) of claim 20, wherein the outer edge (AR; AR′) is at least partially formed by an inner contour (IK; IK′) of a guide portion (FA; FA′) of the surgical template (OS; OS′).

22. The surgical template (OS; OS′) of claim 20, wherein:
the template body (SK; SK′) comprises an attachment portion (BA; BA′) on which a plurality of through-openings (DO) extend from the outer face (AS) to the inner face (IS); and
each of the plurality of through-openings (DO) is configured for guiding a respective attachment mechanism therethrough for attaching the surgical template (OS; OS′) to the temporal bone.

23. The surgical template (OS; OS′) of claim 22, wherein:
the outer edge (AR; AR′) is at least partially formed by an inner contour (IK; IK′) of a guide portion (FA; FA′) of the surgical template (OS; OS′);
the attachment portion (BA; BA′) at least partially peripherally surrounds the guide portion (FA; FA′); and
the guide portion (FA; FA′) projects toward the outer face (AS) with respect to the attachment portion (BA; BA′) or the attachment portion (BA) steplessly transitions into the guide portion (FA).

24. The surgical template (OS′) of claim 20, wherein the template body (SK′) comprises a cover portion (AA) that forms a partition (TW), the partition (TW) separating the outer face (AS) from the inner face (IS) proximate the cover portion (AA), the partition (TW) configured for at least predominant coverage of an auditory canal (GG′) by the surgical template (OS′).

25. The surgical template (OS′) of claim 24, wherein the cover portion (AA) is curved toward the inner face (IS) and configured for insertion into the auditory canal (GG′), the cover portion (AA) further comprising a side wall (SW) that projects toward the outer face (AS) with respect to the partition (TW).

26. The surgical template (OS′) of claim 24, wherein the cover portion (AA) of the template body (SK′) is disposed at an end (E) with which the template body (SK′) is tilted toward the inner face (IS), the cover portion (AA) defines a portion of the outer edge (AR′) of the opening (D′) by an edge (K) of the partition (TW), and

27. The surgical template (OS′) of claim 26, wherein the cover portion (AA) forms a surface (A) configured for placement of a surgical tool to be used to remove the bone part of the temporal bone (SB′) and defines a resection depth.

28. The surgical template (OS′) of claim 26, wherein:
the outer edge (AR; AR′) is at least partially formed by an inner contour (IK; IK′) of a guide portion (FA; FA′) of the surgical template (OS; OS′); and
the outer edge (AR′) of the opening (D′) is formed remote from the tilted end (E) by the inner contour (IK′) of the guide portion (FA′).

29. The surgical template (OS) of claim 20, wherein at least one drill guide portion (BFA) is formed on the template body (SK), each of the at least one drill guide portion (BFA) forming at least one drill hole (BK) extending from the outer face (AS) to the inner face (IS), the at least one drill hole (BK) configured for guiding a bone drill (KB) for removing a bone part of the temporal bone (SB) in each case.

30. The surgical template (OS) of claim 29, wherein each of the at least one drill guide portion (BFA) comprises a guide region (FB) at least on an end situated on the outer face (AS), an inner diameter of the drill hole (BK) approximated to an outer diameter of the bone drill (KB) in the guide region (FB).

31. The surgical template (OS) of claim 30, wherein each guide region (FB) is formed by a guide sleeve (FH) that is introduced into the template body (SK) on the at least one guide portion (BFA).

32. The surgical template (OS) of claim 29, wherein a stop surface (ASF) is formed on the at least one drill guide portion (BFA) flush against an opening in the drill hole (BK) on the outer face, the stop surface (ASF) forming a stop for a surgical tool guiding the bone drill (KB), a length of the respective drill hole (BK) corresponding to a drill depth which is to be achieved via the bone drill (KB) on the inner face (IS) when the surgical tool has reached the respective stop surface (ASF).

33. The surgical template (OS) of claim 29, wherein a plurality of drill guide portions (BFA) is formed.

34. The surgical template (OS) of claim 29, wherein:
the outer edge (AR; AR′) is at least partially formed by an inner contour (IK; IK′) of a guide portion (FA; FA′) of the surgical template (OS; OS′); and
the at least one drill guide portion (BFA) is formed on the guide portion (FA) adjoining the inner contour (IK).

35. The surgical template (OS; OS′) of claim 20, wherein the template body (SK; SK′) is designed in a patient-specific manner.

36. The surgical template (OS; OS′) of claim 35, wherein the template body (SK; SK′) comprises a contact contour (AK; AK′) on the inner face (IS), the contact contour (AK; AK′) configured at least approximately as a counterpart to a surface contour of the temporal bone (SB; SB′) in the placement region.

37. A method for configuring and/or manufacturing a surgical template (OS; OS′) for use proximate a temporal bone (SB; SB′), the method comprising:
forming a template body (SK; SK′) with an outer face (AS) and an inner face (IS), the template body (SK; SK′) placeable on the temporal bone at the inner face (IS), wherein an opening (D; D′) extends from the outer face (AS) to the inner face (IS) in the template body (SK; SK′), and an outer edge (AR; AR′) is defined on the opening (D; D′), the outer edge (AR; AR′) corresponding to an outer contour of a bone part of the temporal bone (SB; SB′) to be removed.

38. The method of claim 37, further comprising configuring and/or manufacturing a temporal bone implant (SI) together with the surgical template (OS; OS′).
